(19) United States
(12) Patent Application Publication
(10) Pub. No.: US 2024/0382160 A1
(43) Pub. Date: Nov. 21, 2024

(54) SYSTEMS AND METHODS FOR BLOOD PRESSURE DEVICE CALIBRATION

(71) Applicant: BaroPace, Inc., Ashland, OR (US)

(72) Inventor: Michael Burnam, Ashland, OR (US)

(73) Assignee: BaroPace, Inc., Ashland, OR (US)

(21) Appl. No.: 18/571,009

(22) PCT Filed: Jun. 16, 2022
(86) PCT No.: PCT/US2022/072989

§ 371 Date: Dec. 15, 2023

Related U.S. Application Data
(60) Provisional application No. 63/212,012, filed on Jun. 17, 2021.

(30) Foreign Application Priority Data
Jun. 10, 2022 (WO) PCT/US2022/072866

(51) Int. Cl. A61B 5/00; A61B 5/021; G16H 10/60

(52) U.S. Cl. CPC A61B 5/7267; A61B 5/02108; A61B 5/6824; A61B 5/684; A61B 5/7203; A61B 5/7225; A61B 5/7246; G16H 10/60; A61B 2560/0228; A61B 2560/0238

(57) ABSTRACT

Techniques and systems for calibrating a blood pressure measuring device are disclosed. They include sensing a first blood pressure by a first device when the first device is at a first height, sensing a second blood pressure by the first device when the first device is at a second height, the second height being different than the first height, and generating the blood pressure calibration factor based on the first blood pressure, the first height, the second blood pressure, and the second height.

### CROSS-REFERENCE TO RELATED APPLICATIONS

This application claims priority to International Application No. PCT/US2022/072866 filed Jun. 10, 2022 which claims priority to U.S. Provisional Application No. 63/212,012, filed on Jun. 17, 2021, the entireties of each of which are incorporated by reference herein. This application also claims direct priority to U.S. Provisional Application No. 63/212,012, filed on Jun. 17, 2021.

### TECHNICAL FIELD

Various embodiments of the present disclosure relate generally to periodic calibration of a blood pressure measuring device, and more particularly, to systems and methods for calibrating blood pressure based on calibration of the blood pressure device.

### INTRODUCTION

Current blood pressure monitoring device based readings can be inaccurate based on the blood pressure device not being calibrated for a given user. For example, blood pressure devices can output a lower blood pressure if raised above the user's heart and a higher blood pressure if lowered below the user's heart. This inaccuracy in blood pressure measurements can cause improper medication dosing, inaccurate diagnoses, among other issues for users and physicians.

The introduction description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

According to certain aspects of the disclosure, methods and systems are disclosed for calibrating a blood pressure measuring device.

In one aspect, an exemplary embodiment of a method for calibrating a blood pressure measuring device may include: sensing a first blood pressure by a first device when the first device is at a first height; sensing a second blood pressure by the first device when the first device is at a second height, the second height being different than the first height; and generating the blood pressure calibration factor based on the first blood pressure, the first height, the second blood pressure, and the second height.

In another aspect, an exemplary embodiment of a system for calibrating a blood pressure measuring device may include: at least one memory storing instructions; and at least one processor executing the instructions to perform a process, the at least one processor configured to: receive a blood pressure sensed using the PPG device; receive a PPG device height when the blood pressure is sensed; and modify the blood pressure based on the PPG device height and a blood pressure calibration factor, wherein the blood pressure calibration factor is based on a first blood pressure sensed at a first device height and a second blood pressure sensed at a second device height.

In another aspect, an exemplary embodiment of a method for calibrating a blood pressure measuring device may include: sensing a first blood pressure using a PPG device, when the PPG device is at a first position; sensing a second blood pressure using the PPG device, when the PPG device is at a second position; determining a blood pressure calibration factor based on the first blood pressure, the first position, the second blood pressure, and the second position; sensing a third blood pressure using the PPG device when the PPG device is at a PPG device position; and modifying the third blood pressure based on the PPG device position and the blood pressure calibration factor.

### BRIEF DESCRIPTION OF THE FIGURES

The accompanying drawings, which are incorporated in and constitute a part of this specification, illustrate various examples and, together with the description, serve to explain the principles of the disclosed examples and embodiments.

Aspects of the disclosure may be implemented in connection with embodiments illustrated in the attached drawings. These drawings show different aspects of the present disclosure and, where appropriate, reference numerals illustrating like structures, components, materials, and/or elements in different figures are labeled similarly. It is understood that various combinations of the structures, components, and/or elements, other than those specifically shown, are contemplated and are within the scope of the present disclosure. Moreover, there are many embodiments described and illustrated herein.

FIG. 1A depicts an exemplary environment for calibrating a blood pressure measuring device using a first blood pressure device as the source of calibration, according to one or more embodiments.

FIG. 1B depicts an example diagram for calibrating a blood pressure measuring device using a first blood pressure device as the source of calibration, according to one or more embodiments.

FIG. 1C depicts another example diagram for calibrating a blood pressure measuring device using a first blood pressure device as the source of calibration, according to one or more embodiments.

FIG. 1D depicts another example diagram of calibrating a blood pressure measuring device using a first blood pressure device as the source of calibration, according to one or more embodiments.

FIG. 2A depicts a flowchart of an exemplary method for calibrating a blood pressure measuring device using a first blood pressure device as the source of calibration, according to one or more embodiments.

FIG. 2B depicts another flowchart of an exemplary method for determining a calibration factor, according to one or more embodiments.

FIG. 3A depicts an example diagram for calibrating a blood pressure measuring device using a photoplethysmography (PPG) device as the source of calibration, according to one or more embodiments.

FIG. 3B depicts another example diagram for calibrating a blood pressure measuring device using a PPG device as the source of calibration, according to one or more embodiments.

FIG. 3C depicts another example diagram for calibrating a blood pressure measuring device using a photoplethysmography device as the source of calibration, according to one or more embodiments.

FIG. 4 depicts another flowchart of an exemplary method for calibrating a blood pressure measuring device using a PPG device as the source of calibration, according to one or more embodiments

FIG. 5 depicts an example of training a machine learning model, according to one or more embodiments.

FIG. 6 depicts an example of a computing device, according to one or more embodiments.

Notably, for simplicity and clarity of illustration, certain aspects of the figures depict the general structure and/or manner of construction of the various embodiments. Descriptions and details of well-known features and techniques may be omitted to avoid unnecessarily obscuring other features. Elements in the figures are not necessarily drawn to scale; the dimensions of some features may be exaggerated relative to other elements to improve understanding of the example embodiments. For example, one of ordinary skill in the art appreciates that the side views are not drawn to scale and should not be viewed as representing proportional relationships between different components. The side views are provided to help illustrate the various components of the depicted assembly, and to show their relative positioning to one another.

### DETAILED DESCRIPTION

Reference will now be made in detail to examples of the present disclosure, which are illustrated in the accompanying drawings. The present disclosure is neither limited to any single aspect or embodiment thereof, nor is it limited to any combinations and/or permutations of such aspects and/or embodiments. Moreover, each of the aspects of the present disclosure, and/or embodiments thereof, may be employed alone or in combination with one or more of the other aspects of the present disclosure and/or embodiments thereof. For the sake of brevity, certain permutations and combinations are not discussed and/or illustrated separately herein. Notably, an embodiment or implementation described herein as “exemplary” is not to be construed as preferred or advantageous, for example, over other embodiments or implementations; rather, it is intended to reflect or indicate the embodiment(s) is/are “example” embodiment(s). Wherever possible, the same reference numbers will be used throughout the drawings to refer to the same or like parts. In the discussion that follows, relative terms such as “about,” “substantially,” “approximately,” etc. are used to indicate a possible variation of ±10% in a stated numeric value.

Aspects of the disclosed subject matter are generally directed to real-time calibration of a blood pressure measuring device (a “device” or a “blood pressure device”) and to correcting for errors in blood pressure due to changes in location and/or heights of the blood pressure measuring device. A blood pressure may be a sensed value, a blood pressure, a sensed value converted into one or more other formats (e.g., by a processor), or the like. A blood pressure may indicate how much pressure a user's blood exerts against the user's artery walls when the user's heart beats (e.g., a systolic blood pressure). A blood pressure may indicate how much pressure a user's blood exerts against the user's artery walls when the user's heart is resting between beats (e.g., diastolic blood pressure).

According to implementations disclosed herein, a first blood pressure may be sensed by a first device (e.g., a blood pressure device, a volumetric device, etc.) when the first device is at a first height or position. The first device may be a gold standard devise as further described herein. The first blood pressure may be a systolic or diastolic blood pressure. A second blood pressure may be sensed by the first device when the first device is at a second height, the second height being different than the first height. A blood pressure calibration factor may be determined based on the first blood pressure, the first height, the second blood pressure, and the second height. The blood pressure calibration factor may be a linear or non-linear relationship, as further discussed herein.

According to an implementation, a third blood pressure may be sensed using a second device (e.g., a device other than the first device, a blood pressure device, a PPG device, etc.), when the second device is at a second device height. The third blood pressure may be modified based on the blood pressure calibration factor and the second device height. For example, the second device height may be applied to the blood pressure calibration factor to determine what amount the third blood pressure is to be modified by.

According to an implementation, the blood pressure measuring device may be a photoplethysmography (PPG) device. The PPG device may be calibrated by first measuring a user's blood pressure using a gold standard blood pressure device, e.g., an arm cuff calibrated against a column of mercury, while the user holds their arm at different locations and/or heights relative to a reference point such as the user's heart. It will be understood that although devices attached to an arm and/or arm positions or locations are generally disclosed herein for simplicity, a device (e.g., a blood pressure device) may be attached to any applicable body part such as, but not limited to, an ankle, an elbow, a bicep, etc., at which a blood pressure can be detected. Accordingly, the subject matter disclosed herein may be applicable to any such body part and is not limited to an arm. The gold standard blood pressure device and may be an invasive or intravascular device. A calibration factor may be determined based on user's blood pressure determined at the different locations and/or heights. The user may use the PPG device to measure blood pressure for the user (e.g., by determining a pulse transmit time) while the user holds their arm at one or more locations and/or heights relative to the user's heart. The blood pressure measured by the PPG device may be calibrated (e.g., adjusted) based on the calibration factor determined based on the gold standard blood pressure device and further based on the respective locations and/or heights identified while measuring the blood pressure by the PPG device. The calibrated values may be output by the PPG device or a processor that receives the measured PPG blood pressure and that applies the calibration factor to the measured PPG blood pressure. Accordingly, embodiments disclosed herein may apply integral trend analysis to eliminate errors introduced by changes in PPG device position(s).

Accordingly, the effect of gravity when measuring a user's blood pressure may be corrected based on the user's individual physiology and/or PPG device position(s), using a calibration factor for the user (e.g., as determined using a gold standard blood pressure measuring device.) A gold standard blood pressure device may be attached to a user's arm. A baseline value of the user's blood pressure may be based on the gold standard blood pressure device measurement when the gold standard blood pressure device is held level with the user's heart. A lower respective blood pressure may be measured when the user holds the arm with the gold standard blood pressure device above the user's heart. Conversely, a higher respective blood pressure may be measured when the user holds the arm with the gold standard blood pressure below their heart. According to techniques disclosed herein, multiple blood pressure, including the baseline value while the user's arm is level with the heart and one or more blood pressure when the user's arm at different locations and/or heights relative to the heart may be recorded. A calibration factor may be determined based on the baseline blood pressure and one or more blood pressure measured at various locations and/or heights relative to the heart. The calibration factor may then apply the location and/or height of a blood pressure device (e.g., a PPG device) to adjust a measured blood pressure. As further disclosed herein, the calibration factor may be based on a linear or non-linear relationship between a blood pressure device location and/or height relative to the user's heart, the ground, or other reference point. Although this disclosure generally discusses locations and/or heights relative to a user's heart, it will be understood that a location and/or height may be relative to any reference point (e.g., the ground, a bed, etc.).

As further disclosed herein, a PPG device or an associated processor or device may utilize a machine learning model to correct for the effect of gravitational forces on the PPG device's measured blood pressure. As discussed in more detail below, in various embodiments, systems and methods are described for using machine learning to correct for the effect of gravitational forces on the PPG device's measured blood pressure. By training a machine learning model, e.g., via supervised, semi-supervised, or unsupervised learning, to learn associations between PPG device location and/or height and blood pressure measurements, the trained machine learning model may be used to correct for the effect of gravitational forces on blood pressure. As discussed herein, there may be numerous benefits to calibrating a PPG device for gravitational changes, such as increased accuracy in medical diagnoses, more effective medical treatments, etc.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. The term “or” is used disjunctively, such that “at least one of A or B” includes, (A), (B), (A and A), (A and B), etc. Relative terms, such as, “substantially” and “generally,” are used to indicate a possible variation of ±10% of a stated or understood value.

It will also be understood that, although the terms first, second, third, etc. are, in some instances, used herein to describe various elements, these elements should not be limited by these terms. These terms are only used to distinguish one element from another. For example, a first contact could be termed a second contact, and, similarly, a second contact could be termed a first contact, without departing from the scope of the various described embodiments. The first contact and the second contact are both contacts, but they are not the same contact.

As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” In addition, the terms “first,” “second,” and the like, herein do not denote any order, quantity, or importance, but rather are used to distinguish an element or a structure from another. Moreover, the terms “a” and “an” herein do not denote a limitation of quantity, but rather denote the presence of one or more of the referenced items.

As used herein, the term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

Terms like “provider,” “medical provider,” or the like generally encompass an entity, person, or organization that may seek information, resolution of an issue, or engage in any other type of interaction with a user, e.g., to provide medical care, medical intervention or advice, or the like. Terms like “user,” “patient,” or the like generally encompass any person (e.g., an individual, a medical provider, etc.) or entity who is using a device, calibrating a device, obtaining information, seeking resolution of an issue, or the like.

As disclosed herein, a gold standard device may be a device used to conduct a gold standard test for calibration. A gold standard test may be a diagnostic test or benchmark that is the best available under reasonable conditions. A gold standard device may be one that has been tested and has a reputation in the field as a reliable method. For example, a gold standard device may include, but is not limited to, a device that uses a column of mercury (e.g., in a cylinder, such as glass) to determine a blood pressure. The gold standard device may detect a force of blood necessary to raise mercury column a known amount at sea level in the Earth's gravitational field. For example, if a catheter is inserted directly into a central artery and the catheter is connected directly to a mercury column, the column may oscillate with the systolic and diastolic blood pressure pumped from the heart a certain number of millimeters of the mercury column. As another example, an inflated cuff around the artery and monitor the oscillations may be a gold standard device. Alternatively, a predicate device calibrated using a device measuring blood pressure with mercury may be a gold standard device (e.g., a cuff device calibrated using a mercury manometer). A gold standard or predicate device may be a Food and Drug Administration (FDA) cleared cuff-type device.

The term “volumetric blood pressure device” or the like generally encompasses devices that obtain and usually record blood pressure at certain intervals, using direct or indirect means of determining pressure. A volumetric blood pressure device may include devices such as a blood pressure cuff, which contains an air bladder which fills up with air and compresses the brachial artery to stop the flow of blood. When the air from the bladder is released, the blood flow restarts. A physician or other medical provider records the systolic and diastolic BP by listening to the flow of blood through a stethoscope. A volumetric blood pressure device may be calibrated against a column of mercury and, thus, may be a gold standard device.

A gold standard device may be implemented by the placement of a catheter into a peripheral artery, most commonly the dorsal metatarsal or femoral artery in smaller patients, although any accessible artery could be used. The catheter may be connected to a pressure transducer with non-compliant tubing filled with heparinized saline to allow continuous measurement, which can be observed on a monitoring device. Such a device may provide continuous values and a pressure waveform that can be observed. The waveform is helpful in assessing pulse quality and pulse deficits caused by an abnormal heart rhythm. Once connected to the patient the transducer may be zeroed to ambient air at the level of the right atrium. This may ensure the readings produced are accurate. Regular flushing of the line may be conducted to ensure patency and accuracy of readings. Arterial catheters require secure taping to prevent hemorrhage due to the catheter becoming dislodged and should be clearly labelled to avoid confusion with intravenous lines.

A gold standard device may be a sphygmomanometer. Such devices may be calibrated over a period of months using a column of mercury as a standard. A gold standard device may be an oscillometric device that can include the use of an inflatable cuff around a limb or tail base, which is attached to a monitor. Measurement may be automatic and may allow for detection of oscillations produced by the artery wall as the cuff deflates.

For intermittent blood pressure measurement, an air-filled occluding cuff can be used that enables blood pressure to be measured either manually or automatically. Manual measurement of blood pressure by an occluding cuff can be done either by palpation or auscultation.

With a palpatory method, an inflatable cuff may be wrapped around the upper arm of a patient. The manometer connected to the cuff by a tube shows the pressure applied. A medical provider feel for a radial pulse, inflates the cuff until the brachial artery collapses, such there is no blood flow any more. The pressure at which a pulse can be detected again while deflating the cuff may correspond to the systolic arterial pressure of the patient. This method may provide a systolic arterial pressure. An auscultatory method may be performed in a similar way, after inflation of the cuff to a pressure above the systolic pressure, sounds can be detected by a stethoscope applied distal of the upper arm cuff during slow deflation. The onset of the sounds may correspond to a patients' systolic arterial pressure, the last sound at decreasing cuff pressure equals the patients' diastolic arterial pressure. An advantage of this technique is that it may provide the diastolic arterial pressure value.

An automated method to measure blood pressure with the help of an occluding cuff may employ an oscillometric technique. A cuff may be inflated to a preset value automatically. Then, the pressure is gradually being reduced. The pressure wave causes oscillations in the vessel, which can be detected by the cuff. Mean arterial pressure corresponds to the maximum of oscillations. An algorithm applied to the change of oscillations may set systolic and diastolic arterial pressure values. The advantages of oscillometry may include the presence of reasonably accurate mean arterial pressure (in normal blood pressure ranges) and the possibility of having an automated tool to determine a patient's blood pressure at a preset interval. The disadvantages may include the overestimation of low and underestimation of high values and the possibility to falsify measurements (e.g., by movement (detected as oscillations) or the patient's arm resting on the bed).

Continuous non-invasive blood pressure monitoring techniques became available that enable a real-time blood pressure curve and numerical blood pressure values to be assessed (just with direct blood pressure measurement).

Continuous non-invasive measurement principles are based on either one of two different techniques, namely arterial applanation tonometry or the volume clamp method. Arterial applanation tonometry may be used to assess mean arterial pressure in the radial artery and allows the calculation of diastolic and systolic arterial pressure (e.g., using population-based algorithms). The technique is used in cardiology to assess central vascular pressures. The pulse wave obtained by applanation tonometry can be analyzed and bears more information than systolic and diastolic pressure alone.

The second technique for non-invasive continuous blood pressure measurement is a volume clamp method (or vascular unloading technology). Blood pressure may be measured at the finger with an inflatable cuff combined with a photodiode. The diameter of the artery in the finger is measured by the photodiode; the pressure in the cuff is adjusted to keep the diameter of the artery constant. From the pressure changes in the cuff, a blood pressure curve can be calculated and transferred to correspond to brachial artery blood pressure.

Oscillometric measurement may be used to monitor systolic, mean and diastolic pressures, unlike the Doppler method which may detect systolic pressure. Single measurements by this method may underestimate arterial pressure by 5-20 mmHg, meaning oscillometric measurement of blood pressure can only be used to observe trends and accuracy may be reduced in patients under 5 kg. Oscillometric monitors have been reported as failing to produce results more often than the Doppler method.

Doppler ultrasonography with the use of an ultrasound probe produces an audible sound of blood flow through an artery. Inflation until no sound may be audible followed by slow deflation of the cuff until sound returns allows determination of systolic pressure. The Doppler method requires more skill due to manual operation and the requirement for detection of the pulse. According to the Doppler method, a spirit swab or clipping the area directly over the artery if required and tolerated, may be implemented, followed by ample ultrasound gel applied to the probe to improve contact. Correct placement can be confirmed by the sound of blood flow, which is typically a “whoosh” sound.

The term “photoplethysmography device” (“PPG” device) may be a light based device and/or may use different techniques to measure the changes in blood flow or volume (e.g., light, pulse transit time measurements, ultrasound, magnetic resonance imaging, indicator dilution methods, intravenous injection of contrast for X-ray imaging, thermography, estimates of capillary filling, etc.). The PPG device may be a wearable device, e.g., a watch, a band, a strap, etc., or a non-wearable device. A PPG device may operate by measuring the “pulse transit time,” which is converted to a respective blood pressure. The pulse transit time measures the time it takes for blood to move from a first part of an artery to a second part of an artery. A PPG device may use a non-invasive optical method for measuring blood volume changes per pulse. A PPG waveform output by a PPG device may represent the mechanical activity of the heart. Blood pressure may be determined by analysis of the PPG waveform. A PPG measurement may be subject to imprecision from a number of factors, including but not limited to, calibration issues, effects on blood pressure based on arm positions (e.g., from the variable contribution of gravity), and/or local vasospasm effects on blood flow such as cold temperature, etc. The blood pressure measurement itself, although improved by the use of light-emitting diodes, suffers inherent drift with prolonged use. For these reasons and more, a means of calibrating a PPG device, and other indirect blood pressure measurement apparatuses not including an oscillometer is disclosed herein.

The term “algorithm,” as used herein, refers to a sequence of defined computer-implementable instructions, typically to solve a class of problems or to perform a computation.

Terms such as “noise,” or the like, as used herein, generally encompass extraneous, irrelevant, or relatively less meaningful data, or any data that is other than a signal intended to be observed. In the case of waveforms, “noise” may include, for example, unwanted signals that are merged with the waveform signal. Terms such as “signal” or the like, a used herein, generally encompass any function that may convey information about a phenomenon. “Signals” may refer to any time varying voltage, current, or electromagnetic wave that carries information or an observable change in a quality, such as quantity, or may refer to the information itself.

As used herein, a “machine learning model” generally encompasses instructions, systems, data, and/or a model configured to receive input, and apply one or more of a weight, bias, classification, or analysis on the input to generate an output. The output may include, for example, a classification of the input, an analysis based on the input, a design, process, prediction, or recommendation associated with the input, or any other suitable type of output. A machine learning model is generally trained using training data, e.g., experiential data and/or samples of input data, which are fed into the model in order to establish, tune, or modify one or more aspects of the model, e.g., the weights, layers, biases, criteria for forming classifications or clusters, or the like. Aspects of a machine learning model may operate on an input linearly, in parallel, via a network (e.g., a neural network), or via any suitable configuration.

The execution of the machine learning model may include deployment of one or more machine learning techniques, such as linear regression, logistical regression, random forest, gradient boosted machine (GBM), deep learning, and/or a deep neural network. Supervised and/or semi-supervised training may be employed. For example, supervised learning may include providing training data and labels corresponding to the training data, e.g., as ground truth. Semi-supervised approaches may include heuristic, generative, low-density, Laplacian or other like models. K-means clustering or K-Nearest Neighbors may also be used, which may be supervised or semi-supervised. Combinations of K-Nearest Neighbors and a semi-supervised cluster technique may also be used. Any suitable type of training may be used, e.g., stochastic, gradient boosted, random seeded, recursive, epoch or batch-based, etc.

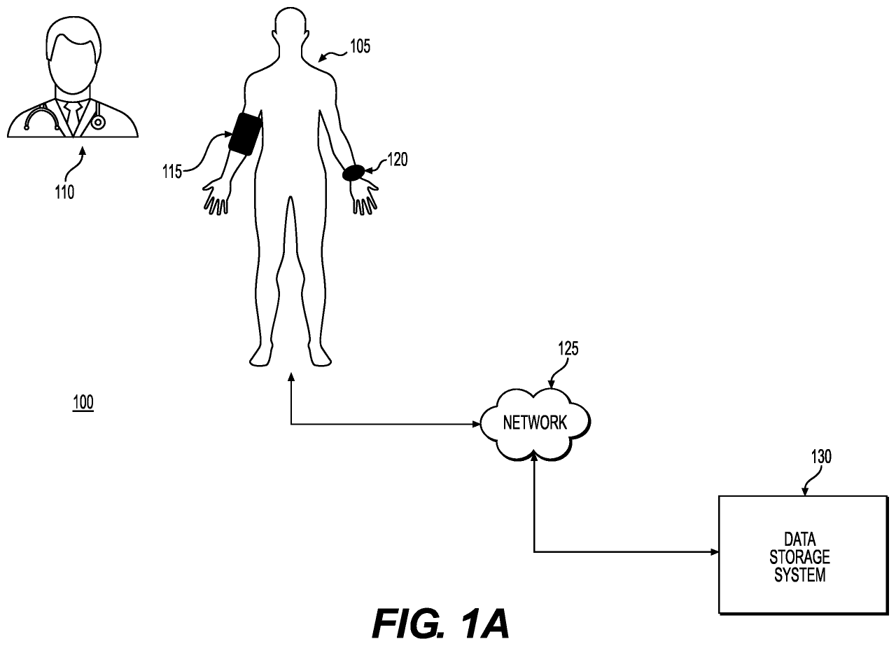

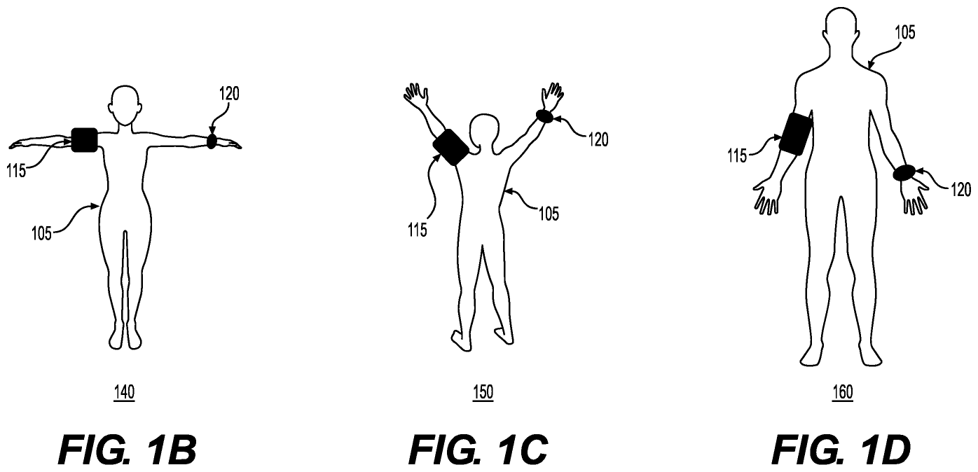

According to implementations of the disclosed subject matter, as shown in the environment 100 of FIG. 1A, a user 105 and/or a medical provider 110 may operate a gold standard blood pressure device 115 and/or a PPG device 120, the results from gold standard blood pressure device 115 and/or PPG device 120 being transmitted via a network 125 to a data storage system 130. User 105 may wear gold standard blood pressure device 115 and PPG device 120 on the same arm simultaneously, on opposite arms simultaneously, or user 105 may wear only one of either gold standard blood pressure device 115 or PPG device 120 at a time.

Gold standard blood pressure device 115 and/or PPG device 120 may operate continuously, at intervals, or at the determination of user 105, medical provider 110, and/or a user. Gold standard pressure device 115 and/or PPG device 120 may include one or more sensors either internal or external to the respective device. The one or more sensors may detective a location or height of the gold standard blood pressure device 115 and/or PPG device 120 relative to a reference point (e.g., a heart, a ground level, etc.) The one or more sensors may include a motion sensor, an accelerometer, an electromechanical sensor, a stadiometer, an active ultrasonic sensor, a passive infrared sensor, a vibration motion sensor, a dual technology or hybrid sensor, a Doppler radar sensor, a tomographic sensor, a gesture detector, a displacement sensor, and/or any other suitable sensor. The one or more sensors may operate continuously, at intervals, or at the instruction or input of user 105, medical provider 110, and/or another user. For example, the one or more sensors may operate while the user moves the arm that has gold standard blood pressure device 115 and/or PPG device 120 attached and not operate (e.g., enter a sleep mode) when the user's arm is not moving. The one or more sensors may operate in response to the activation of gold standard blood pressure device 115 and/or PPG device 120. For example, the one or more sensors may remain idle until gold standard blood pressure device 115 and/or PPG device 120 begins measuring a blood pressure, at which point the one or more sensors may activate. The one or more sensors may determine a gold standard blood pressure device 115 and/or PPG device 120 height and/or positioning for a given blood pressure measurement based on the average position of gold standard blood pressure device 115 and/or PPG device 120 during a time period (e.g., approximately three seconds) that a current blood pressure is being measured. The one or more sensors may determine gold standard blood pressure device 115 and/or PPG device 120 height and/or positioning at a predetermined time over a duration of measuring the current blood pressure (e.g., at the end of a measurement time period, middle of the measurement time period, etc.).

According to an embodiment of the disclosed subject matter, PPG device 120 may include one or more multi-beam light emitting diodes (LEDs). The one or more multi-beam LEDs may be used to detect a level, percent, or amount of one or more hemoglobin subtypes. Certain medication and/or presence of carbon monoxide may increase alternative hemoglobin content (e.g., methemoglobin or sulfhemoglobin). The presence of such alternative hemoglobin content my modify a PPG signal and may also modify pulse oximeter readings. Accordingly, a calibration factor, as disclosed herein, may account for the presence of such alternative hemoglobin content to calibrate blood pressure values. According to an embodiment, an initial calibration may include offsets that may be applied upon detection of such alternative hemoglobin content. Further, a calibration factor, as disclosed herein, may calibrate blood pressure readings upon detection of such alternative hemoglobin content. As disclosed herein, a machine learning model that is trained based on a calibration factor and/or applies a calibration factor may receive, as an input, additional user information. The additional user information may include information based on a user's medical and/or medication information and/or history, a user demographic (e.g., an age, a gender, a body weight, a body mass index (BMI), a height, an arm length, an ethnicity, etc.) a climate, hemoglobin subtypes, or the like.

According to embodiments disclosed herein, one or more forms of noise found in one or more signals and/or values, e.g., volumetric data signals, calibrated blood pressures, etc., may be filtered (e.g., reduced, modified, and/or removed). The one or more signals may be generated using gold standard blood pressure device 115 and/or PPG device 120. A noise reduction algorithm may be used to filter noise. The type of noise reduction algorithm may depend on the type of noise in the data, the type of data, or the like. The noise reduction algorithm may be automatically selected and/or applied or may be selected and/or applied based on user input. A type of noise may include, but is not limited to, high frequency noise, movement noise, and/or any other form of noise. A plurality of noise reduction algorithms may be used to filter noise for a given signal. According to embodiments disclosed herein an amplification may be applied to amplify a signal generated at gold standard blood pressure device 115 and/or PPG device 120. Such signal amplification may be performed prior to, in conjunction with, or post filtering the signal for noise.

According to an embodiment, PPG device 120 blood pressure values may be calibrated using a calibration factor, as disclosed herein, prior to filtering and/or amplification of the corresponding values. Accordingly, PPG device 120 may generate blood pressure signals to determine a blood pressure value for a user. The blood pressure signals may be raw signals or signals based on raw signals detected by PPG device 120. The blood pressure signals may be calibrated using a calibration factor, as discussed herein. The noise reduction algorithm, other filter, and/or an amplification may be applied to the calibrated blood pressure signals (e.g., to generate blood pressure values). Applying the noise reduction algorithm, other filter, and/or an amplification to calibrated blood pressure signals may allow for continuous blood pressure outputs, as further discussed herein. In this embodiment, the calibration factor may be applied to signals output by PPG device 120. Accordingly, the calibration factor may include calibration relationships for signals generated by PPG device 120 and filtering/amplification may be applied to signals already calibrated based on the calibration factor.

A machine learning model may be used to output a calibration factor, to modify the calibration factor, and/or to apply the calibration factor. The machine learning model may analyze data received from user 105, provider 110, gold standard blood pressure device 115, PPG device 120, data storage system 130, and/or any other person, entity, or device. For example, data from gold standard blood pressure device 115 (e.g., mercury column calibrated data) and/or device from PPG device 120 (PPG device data) may be input into the machine learning model. The trained machine learning model may correlate the PPG device 120 height with the current blood pressure. Using this correlation, the machine learning model may use the calibration factor to calibrate the current blood pressure. The machine learning model may output a calibrated current blood pressure.

In some embodiments, the machine learning model may receive additional data, e.g., medical, demographic, or personal information for user 105, etc. For example, in some embodiments, provider 110 may specify a particular condition or characteristic of interest for user 105 that could affect the blood pressure for the user. In some embodiments, the machine learning model may analyze the gold standard data, the PPG device data, and/or any additional data. The machine learning model output may be individualized to a user 105. The individualization may be based on data from a user's or a plurality of users' user height, a user medical conditions, or a user PPG device height relative to a user heart. Training the machine learning model for individualization is discussed in further detail below. In some embodiments, machine learning model output by the machine learning model may be transmitted to user 105, provider 110, gold standard blood pressure device 115, PPG device 120, and/or data storage system 130. In some embodiments, the machine learning data may include medical, diagnostic, or other health-related information or results.

In various embodiments, a processor or storage component, gold standard blood pressure device 115, and/or PPG device 120 may generate, store, train, or use the machine learning model and/or may include instructions associated with the machine learning model, e.g., instructions for generating the machine learning model, training the machine learning model, using the machine learning model, etc. For example, blood pressure measured using gold standard blood pressure device 115 may be transmitted, via a Bluetooth protocol, to processor associated with PPG device 120. The processor may receive the measured blood pressure to determine a calibration factor (e.g., output by a machine learning model accessible by the processor). The processor or one or more other processors may apply the calibration factor to current blood pressure measured by the PPG device, based on corresponding heights and/or locations of the PPG device during respective measurements.

In some embodiments, a system or device other than gold standard blood pressure device 115 or PPG device 120 may be used to generate and/or train the machine learning model. For example, such a system may include instructions for generating the machine learning model, the training data and ground truth, and/or instructions for training the machine learning model. A resulting trained machine learning model may then be provided to PPG device 120 or a component associated with PPG device 120 such that the trained machine learning model can output a calibration factor, modify the calibration factor, and/or apply the calibration factor.

Generally, a machine learning model includes a set of variables, e.g., layers, nodes, neurons, filters, weights, biases, etc., that are tuned, e.g., weighted or biased, to different values via the application of training data. In supervised learning, e.g., where a ground truth is known for the training data provided, training may proceed by feeding a sample of training data into a model with variables set at initialized values, e.g., at random, based on Gaussian noise, a pre-trained model, or the like. The output may be compared with the ground truth to determine an error, which may then be back-propagated through the model to adjust the values of the variable.

Training may be conducted in any suitable manner, e.g., in batches, and may include any suitable training methodology, e.g., stochastic or non-stochastic gradient descent, gradient boosting, random forest, adaptive moment estimation (“ADAM”), etc. Training may be conducted with or without sample and/or class weighting. In some embodiments, a portion of the training data may be withheld during training and/or used to validate the trained machine-learning model, e.g., compare the output of the trained model with the ground truth for that portion of the training data. The training of the machine learning model may be configured to cause the machine learning model to learn associations between (i) gold standard data and/or PPG device data and (ii) gravitational effects based on device positioning, such that the trained machine learning model is configured to determine an output (e.g., corrected PPG device data) in response to the input data based on the learned associations. For example, the machine learning model may receive PPG device data points (e.g., blood pressure) associated with a particular arm positioning, which the machine learning model may be trained to correct based on the calibration factor applied to the arm positioning.

In various embodiments, the variables of a machine learning model may be interrelated in any suitable arrangement in order to generate the output. For example, in some embodiments, the machine learning model may include architecture that is configured to identify, isolate, and/or extract features, geometry, and or structure in input data. For example, the machine learning model may include one or more convolutional neural networks (“CNN”) configured to identify features in the signal-processed data, and may include further architecture, e.g., a connected layer, neural network, etc., configured to determine a relationship between the identified features in order to determine a location in the signal-processed data.

In some embodiments, the network 125 may connect one or more components of the environment 100 via a wired connection, e.g., a USB connection between gold standard blood pressure device 115 and PPG device 120. In some embodiments, the network 125 may connect one or more aspects of the environment 100 via an electronic network connection, for example a Bluetooth connection, a wide area network (“WAN”), a local area network (“LAN”), personal area network (“PAN”), or the like. In some embodiments, the electronic network connection includes the Internet, and information and data provided between various systems occurs online. “Online” may mean connecting to or accessing source data or information from a location remote from other devices or networks coupled to the Internet. Alternatively, “online” may refer to connecting or accessing an electronic network (wired or wireless) via a mobile communications network or device. The Internet is a worldwide system of computer networks-a network of networks in which a party at one computer or other device connected to the network can obtain information from any other computer and communicate with parties of other computers or devices. The most widely used part of the Internet is the World Wide Web (often-abbreviated “WWW” or called “the Web”). A “website page,” a “portal,” or the like generally encompasses a location, data store, or the like that is, for example, hosted and/or operated by a computer system so as to be accessible online, and that may include data configured to cause a program such as a web browser to perform operations such as send, receive, or process data, generate a visual display and/or an interactive interface, or the like. According to embodiments, environment 100 may be a closed loop such that no external network connection may be necessary to implement the techniques disclosed herein. The closed loop maybe used to provide a real-time automatic method that is self-contained and not dependent upon linkage to a remote server containing additional software, often referred to as “edge computing.” The method is also suitable for transmission to the cloud to allow for an interface with conventional electronic health records and other data analysis and reporting processes.

In such a closed loop system, for example, the gold standard blood pressure device 115 may transmit blood pressure and/or a calibration factor to PPG device 120 over a Bluetooth connection. PPG device 120 may be associated with a processor that may apply the received blood pressure and/or calibration factor from the gold standard blood pressure device 115 to generate calibrated blood pressure outputs based on blood pressure and respective device heights and/or locations of the PPG device 120 (e.g., using a machine learning model). Accordingly, the connections within the environment 100 can be wireless, wired, or be any other suitable connection, or any combination thereof.

In some embodiments, the data storage system 130 may store the data from and/or provide data to various aspects of the environment 100. Data storage system 130 may include a server system, an electronic medical data system, computer-readable memory such as a hard drive, flash drive, disk, etc. In some embodiments, data storage system 130 includes and/or interacts with an application programming interface for exchanging data to other systems, e.g., one or more of the other components of the environment 100. Data storage system 130 may include and/or act as a repository or source for data from gold standard blood pressure device 115, data from PPG device 120, medical history and/or diagnoses for user 105, machine learning data, and/or other forms of data. Data storage system 130 may be external to or may be a part of gold standard blood pressure device 115 or PPG device 120.

According to implementations of the disclosed subject matter, as shown in the environment 140 of FIG. 1B, environment 150 of FIG. 1C, and environment 160 of FIG. 1D, user 105 or medical provider 110 may operate gold standard blood pressure device 115 and/or PPG device 120 while user 105 holds their arm(s) at different positions to calibrate PPG device 120 for use by user 105. Although these figures show both the gold standard blood pressure device 115 and PPG device 120 on user 105 at the same time, it will be understood that a first set of data (e.g., blood pressure for calibration) may be collected while gold standard blood pressure device 115 is attached to the user at a first time. Subsequently, once blood pressure for calibration using the gold standard blood pressure device 115 are recorded, the user may remove the gold standard blood pressure device 115 and wear the PPG device 120 on the same arm or the other arm as the gold standard blood pressure device 115.

In an example, user 105 may first wear gold standard blood pressure device 115 such that gold standard blood pressure device 115 is level with the user's heart in order to determine a first calibration blood pressure as shown in FIG. 1B. The first calibration blood pressure may be determined by, for example, pressing a button or otherwise activating gold standard blood pressure device 115. As shown in FIG. 1C, a second calibration blood pressure may be determined while user 105 holds their arm at a second position (e.g., above their heart). As shown in FIG. 1D, a third calibration blood pressure may be determined while user 105 holds their arm at a third position (e.g., below their heart). The first, second, and third calibration blood pressure may be stored (e.g., in a memory or at data storage system 130).

Calibration blood pressure may be determined using gold standard blood pressure device 115 as few or as many times as necessary. The number of times may be based on the quality of the calibration blood pressure. The quality of the calibration blood pressure may be determined using a process to analyze the calibration blood pressure after one or more filters are applied to the values (e.g., a noise filter, an amplifier, etc.). Although a first, second, and third position are shown in FIGS. 1B-1D, any number of calibration blood pressure greater than two may be collected at respective heights and/or locations. As discussed herein, user 105 may attach gold standard blood pressure device 115 and/or PPG device 120 to the same arm, to opposite arms, or may wear one of the devices at a time to calibrate and/or operate PPG device 120.

The calibration blood pressure may be used to generate a calibration factor. The calibration factor may be generated at gold standard blood pressure device 115, PPG device 120, or an external component (e.g., a processor, a cloud component, etc.). The calibration factor may be based on a linear or non-linear relationship between the plurality of calibration blood pressure and the corresponding positions of the gold standard blood pressure device 115 relative to a reference point (e.g., the user's heart, a ground level, etc.). The calibration factor may be used to correct PPG device data for the influence of gravity on blood pressure when the arm of user 105 is held at different heights or positions. For example, user 105 may wear PPG device 120 and one or more current blood pressure may be generated using PPG device 120. The calibration factor may be applied to the one or more current blood pressure based on the respective location and/or height of the PPG device during detection of the one or more current blood pressure. The current blood pressure may be adjusted to generate respective one or more calibrated current blood pressure that are calibrated based on the calibration factor and the respective heights and/or locations of the PPG device. The calibrated current blood pressure may be provided as an output from the PPG device 120. The calibrated current blood pressure may be provided via a display or to one or more components of environment 100 or an external component (e.g., via a Bluetooth or other network 125 connection).

According to an embodiment of the disclosed subject matter, PPG device 120 may be initially calibrated prior to use by a user. PPG device 120 may be initially calibrated by a manufacturer before packaging or after packaging PPG device 120 (e.g., a factory calibration). For example, PPG device 120 may be manufactured and packaged, and an initial calibration update may be provided to PPG device 120 wirelessly. Alternatively, PPG device 120 may be initially calibrated prior to use of the device by user. For example, as part of the setup, a software update that includes an initial calibration for PPG device 120 may be installed.

The initial calibration may be based on population-based data that includes the most likely blood pressure(s) to correspond to given PPG signal(s) generated by PPG device 120. Accordingly, the initial calibration may be used to determine blood pressure values based on senor based signals generated or detected by PPG device 120. The initial calibration may map or otherwise provide a relationship of blood pressure values to sensor signals sensed by PPG device 120. The initial calibration may allow PPG device 120 to output (e.g., via a display or an external component) initially calibrated blood pressure values based on the sensed signals.

This initial calibration may correspond to a neutral location or position of PPG device 120 (e.g., relative to a user's heart), such that the initial calibration may not account for different locations or positions of PPG device 120 in use, as discussed herein.

Accordingly, the initial calibration may not take into consideration an arm length (e.g., the distance between PPG device 120 on the arm and the level of the heart), an overall PPG device 120 position relative to a reference point (e.g., an arm position relative to a user's heart), and/or differences between population groups, age, gender, medication, or the like. For instance, an anti-hypertensive drug that functions on the basis of vasodilatation may change vascular tone. Such a change may, for example, exaggerate the changes associated with arm position as the blood column in a more relaxed artery may contain more blood with an arm having PPG device 120 below a reference point (e.g., due to additional weight). Accordingly, the initial calibration may not be adequate even if a PPG device 120 position is known, as further discussed herein. According to an embodiment, prior to use, PPG device 120 may be additionally calibrated in accordance with the techniques disclosed herein. The additional calibration may be specific to a given user that uses PPG device 120. The additional calibration may be conducted once, prior to use of PPG device 120 and/or may be calibrated periodically, prior to each use of PPG device 120, or at any other frequency as determined by a user, PPG device 120, or medical provider. For example, PPG device 120 may generate an alert or notification when a given additional calibration is outdated (e.g., if a threshold amount of time has passed), is no longer accurate (e.g., as determined by a machine learning model based on, for example, changes in additional user information), or the like. Accordingly, the calibration may be conducted for a moment in time for a defined period of time (e.g., for a period of time while the user's medication or treatment is constant). For simplicity, an additional calibration is generally referenced to as a “calibration,” herein.

As disclosed herein, a calibration may be used to generate a calibration factor that accounts for a PPG device 120 position and/or additional user information. PPG device 120 may be calibrated against or using a gold device or predicate device at a user site (e.g., a user's home or work) or at another facility (e.g., a health care provider facility). A user may use the gold standard or predicate device (gold standard blood pressure device 115) to determine blood pressure values at various arm positions for an arm that gold standard blood pressure device 115 is attached to. Similarly, a user may use PPG device 120 to determine blood pressure values at the various arm positions. PPG device 120 blood pressures may be determined at the same time as gold standard blood pressure device 115 blood pressures (e.g., if PPG device 120 is attached to the same arm as gold standard blood pressure device 115 while determining the respective blood pressures). The blood pressure values from gold standard blood pressure device 115 may be provided to PPG device 120 (e.g., automatically via wireless or wired connections or via user input) or to another processor in communication with PPG device 120. As disclosed herein, a calibration factor to calibrate or correct PPG device 120 blood pressure values may be determined to correct or individualize a calibration offset for PPG device 120, specifically for the user. Such a calibration may result in use of PPG device 120 as a medical grade device and may result in more accurate blood pressure value outputs than a consumer device.

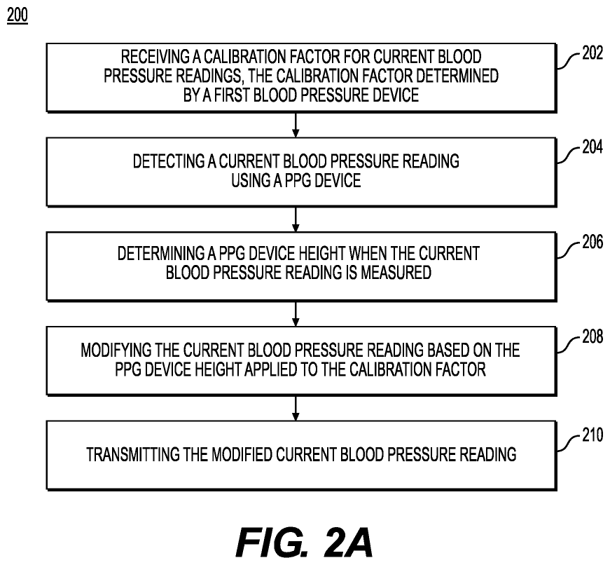

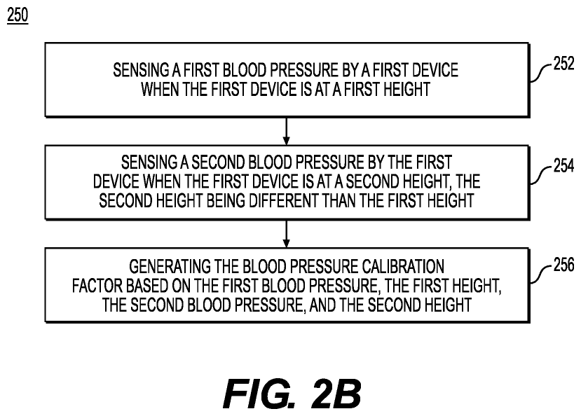

FIG. 2A illustrates an exemplary process for calibrating PPG device 120 blood pressure based on calibration using gold standard blood pressure device 115. At step 202, PPG device 120 receives a calibration factor. As discussed herein, the calibration factor may be determined by using a first device (e.g., gold standard blood pressure device 115) to determine a first calibration blood pressure when the first device is at a first height and a second calibration blood pressure when the first device is at a second height. The calibration blood pressure may be provided from the first device to PPG device 120 and/or another component via a wired or wireless connection, as disclosed herein. The calibration blood pressure may be provided, for example, using Bluetooth Low Energy (BLE) or other communication means. The respective calibration blood pressure and heights may be used to generate a calibration factor (e.g., a linear or non-linear relationship) associating differences in blood pressure based on height, as discussed above. The calibration factor may be generated using a machine learning model or may be used to train a machine learning model. The machine learning model may be configured to generate a calibration factor based on the respective calibration blood pressure and heights and/or on additional user information such as information based on a user's medical and/or medication information and/or history, a user demographic (e.g., an age, a gender, a body weight, a body mass index (BMI), a height, an arm length, an ethnicity, etc.) a climate, hemoglobin subtypes, or the like.

The calibration factor may be determined by an algorithm configured to receive the calibration blood pressure and respective heights, and/or additional user information, to generate the calibration factor. Accordingly, the algorithm may perform calculations using pre-programmed decision analysis using predefined mathematical relationships. The calibration factor may be the linear or non-linear relationship or an analyzed version of the linear or non-linear relationship. For example, the calibration factor may be a variable algorithm that is the best fit to the calibration blood pressure as a function of height. The calibration factor may be provided to PPG device 120 such that the calibration factor is stored at PPG device 120 and/or a component (e.g., data storage system 130) accessible by PPG device 120.

At step 204, PPG device 120 may detect a current blood pressure for user 105. The current blood pressure may be stored in memory or at a component (e.g., data storage system 130) accessible by PPG device 120. At step 206, one or more sensors may be used to determine the height and/or position of PPG device 120 relative to a reference point, e.g., PPG device 120's position relative to user 105's heart. The PPG device 120 location and/or height may be stored in memory or at a component (e.g., data storage system 130) accessible by PPG device 120. The PPG device 120 location and/or height may be stored or accessible by the same storage or component as the current blood pressure. Similarly, the calibration factor may be stored or accessible by the same storage or component as the current blood pressure and the PPG device 120 location and/or height.

At step 208, the current blood pressure from step 204 may be calibrated using the calibration factor from step 202 and the PPG device 120 location and/or height corresponding to the current blood pressure. For example, the calibration factor may be applied to the PPG device 120 location and/or height corresponding to the current blood pressure. The output of the application may be an adjustment factor by which the current blood pressure is to be adjusted. Alternatively, the calibration factor may be in the form of a trained machine learning model (e.g., a non-linear calibration factor). According to this implementation, the machine learning model may be trained based on the calibration blood pressure and respective device height and/or locations of the first device. The machine learning model may be configured to output calibrated blood pressure based on current blood pressure and device height and/or locations of PPG device 120. The calibrated blood pressure may be output at step 210.

As disclosed herein, the calibration factor may be applied to blood values before application of a filter or amplification. For example, at 208, current blood pressure from step 204 may be calibrated using the calibration factor from step 202. After the calibration, one or more filters (e.g., noise reduction filters) or amplifications may be applied to the calibrated current blood pressure. Such post calibrated filtering or amplification may allow for continuous blood pressure outputs from PPG device 120, as detected blood pressures are calibrated automatically and additional blood pressures are continuously detected by PPG device 120, without waiting for filtering or amplification.

According to implementations disclosed herein, as shown in flow chart 250 of FIG. 2B, a first blood pressure may be sensed by a first device (e.g., a blood pressure device, a gold standard device, etc.) when the first device is at a first height or position at 252. The first blood pressure may be a systolic or diastolic blood pressure. At 254, a second blood pressure may be sensed by the first device when the first device is at a second height, the second height being different than the first height. At 256, a blood pressure calibration factor may be determined based on the first blood pressure, the first height, the second blood pressure, and the second height. The blood pressure calibration factor may be a linear or non-linear relationship, as further discussed herein.

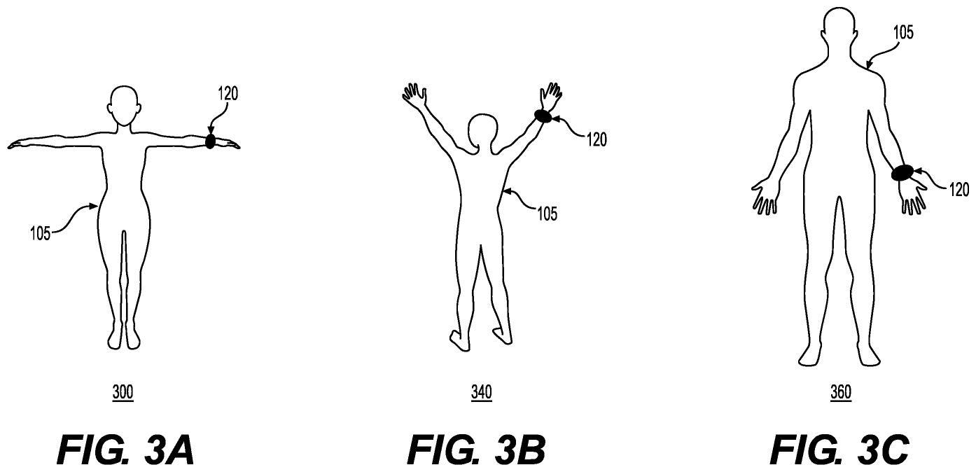

According to implementations of the disclosed subject matter, PPG device 120 may be configured to output calibrated blood pressure without using the first device (e.g., the gold standard blood pressure device 115). Environment 300 of FIG. 3A, environment 340 of FIG. 3B, and environment 360 of FIG. 3C, show PPG device 120 while user 105 holds their arm(s) at different positions, to calibrate PPG device 120.

PPG device 120 may be used to determine calibration blood pressure during a calibration period. Alternatively, as further discussed herein, PPG device 120 may calibrate blood pressure without first calibrating PPG device 120. According to an implementation of using a calibration period, user 105 may operate PPG device 120 while user 105 holds PPG device 120 level with the user's heart in order to determine a first calibration blood pressure, as shown in FIG. 3A. A calibration blood pressure may be determined by pressing a button or otherwise activating PPG device 120. As shown in FIG. 3B, the user may operate PPG device 120 at a second time to determine a second calibration blood pressure at a second position of PPG device 120 (e.g., above the user's heart). As shown in FIG. 3C, the user may operate PPG device 120 at a third time to determine a third calibration blood pressure at a third position of PPG device 120 (e.g., below the user's heart).

The calibration blood pressure and respective PPG device 120 positions may be used to generate a calibration factor. The calibration factor may be generated at PPG device 120, or different device component configured to receive the calibration blood pressure and respective PPG device 120 positions, in accordance with the techniques disclosed herein.

The calibration factor may be used to modify current blood pressure collected using PPG device 120 after the calibration period. For example, the calibration factor may include a relationship between PPG device 120 positions and adjustments to corresponding current blood pressure. According to this example, the calibration factor-based relationship may be a percentage increase or decrease in a blood pressure, based on the position of PPG device 120 at the time of determining the reading. For example, a distance of two feet above a user's heart may correspond to a three percent increase in the corresponding current blood pressure, according to the user's calibration factor for PPG device 120. The user may use PPG device 120 while exercising. A current blood pressure while exercising with the PPG device 120 two feet above the user's heart may be multiplied by 1.03 to generate a calibrated blood pressure. It will be understood that the absolute blood pressure value change based on a three percent increase while exercising may be a greater than the absolute blood pressure value change of a similar three percent increase (based on PPG device 120 being two feet above the user's heart), while the user is resting. Accordingly, the calibration factor may be used to apply the relationship based on PPG device 120 position. However, the absolute value of change may defer based on a current blood pressure.

According to this implementation, PPG device 120 may be calibrated at a first time (e.g., at calibration conditions such as during rest). A calibration factor may be determined based on calibration blood pressure determined during the first time. Subsequently, the calibration factor may be applied to current blood pressure determined by PPG device 120, to adjust the current blood pressure to calibrated blood pressure.

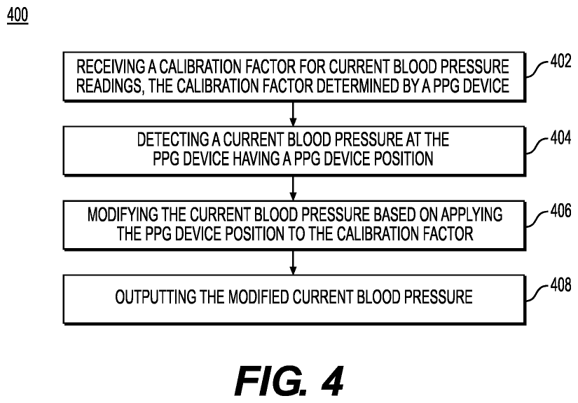

FIG. 4 depicts an exemplary process for calibrating PPG device 120 blood pressure based on the calibration factor discussed above. At step 402, PPG device 120 may determine a calibration factor. The calibration factor may be determined by first: measuring a baseline blood pressure of a user based on PPG device 120 being level with the user's heart, measuring a first blood pressure of the user based on PPG device 120 being at a first position relative to the user's heart, and measuring a second blood pressure of the user based on PPG device 120 being at a second position relative to the user's heart. The calibration factor may then be determined by linearly or non-linearly associating the baseline blood pressure, the first blood pressure, the second blood pressure, and the respective positions of PPG device 120. The calibration blood pressure may be recorded when the user is in a resting state.

At step 404, PPG device 120 may determine a current blood pressure of user 105 while the height and/or position of PPG device 120 relative to the ground and/or heart of user 105 is also determined. At step 406, the current blood pressure from step 404 may be modified based on the calibration factor from step 402 to generate a calibrated current blood pressure. At step 408, PPG device 120 may output the calibrated current blood pressure. PPG device 120 may store the calibrated current blood pressure at data storage system 130 and/or at another component, and/or may transmit the calibrated current blood pressure to another entity or device, e.g., a medical provider's user portal.

According to an implementation of the disclosed subject matter, PPG device 120 may include or have access to a calibration factor. The calibration factor may be stored at PPG device 120 or a component accessible by PPG device 120 during a manufacturing process or during a system update. Accordingly, PPG device 120 may determine one or more current blood pressure of a user. The height and/or position of PPG device 120 may be determined when PPG device 120 determines the respective one or more current blood pressure. The one or more current blood pressure may be modified based on the calibration factor and the corresponding height and/or positions of PPG device 120, in accordance with the techniques disclosed herein. According to this implementation, a user may not calibrate PPG device 120. Additionally, as disclosed herein, a machine learning model may update the calibration factor for PPG device 120 based on user data and/or data from one or more other users that is provided to the machine learning model.

As disclosed herein, a machine learning model may generate a calibration factor based on historical data and/or user data. The machine learning model or a second machine learning model may update the calibration factor based additional user information or data from other users (e.g., a cohort of other users that may have overlapping attributes with a given user). For example, a calibration factor may be a linear or non-linear relationship of blood pressure and PPG device 120 height and/or position relative to a reference point. A machine learning model may update the calibration factor over time, based on additional user information (e.g., medical and/or medication information and/or history, a user demographic, hemoglobin information, a climate, etc.). The machine learning model may receive the user data and may apply weights, layers, biases, etc., to determine adjustments to the calibration factor for the given user. The updates may be based on inherent changes to the user's anatomy or medical condition(s) such that, over time, an original calibration factor may not be as applicable to the user as an updated calibration factor.

According to implementations of the disclosed subject matter, PPG device 120 may generate a continuous output signal (e.g., blood pressure). The continuous signal may include a string of sine wave and/or sine wave like outputs that are available for motion and/or noise correction or signal amplification. Upon correction and/or amplification, a number of cycles of the signal may be averaged or otherwise manipulated for discrete measurement of blood pressure. The number of cycles may correspond to approximately 3 seconds per sample or less, which may result in a continuous blood pressure output using PPG device 120.

Changes in PPG device 120 positions (e.g., due to arm movement) within a sampling period may result in a smeared average result. For example, if an average heart rate for a user is 70 beats per minute, then 3-seconds of sampling time is roughly equal to 4 heart beats or 4 PPG cycles. During the 3-second period, the arm may change position such as if the arm swings in an arc. If this occurs, the 3-second period may not result in a discrete measurement, but rather a smeared measurement (e.g., a smeared average). To prevent smeared measurements, PPG device 120 may be calibrated to detect continuous blood pressure values such that effects of movement are minimized. According to an embodiment, the initial calibration disclosed herein may be used to configure PPG device 120 to provide a continuous output by initially calibrating PPG device 120 against a true continuous blood pressure output, such that every PPG cycle is initially calibrated. For the initial calibration, an invasive blood pressure detection and calibration method, where an arterial cannula is inserted into the central artery of a user, may be used to generate a continuous blood pressure output. This continuous blood pressure output using an invasive device may be used to initially calibrate PPG device 120. A calibration factor, as disclosed herein, may be applied to such a continuous initial calibration, such that PPG device 120 may output continuous calibrated blood pressures based on discrete PPG cycles.

According to an implementation, as an alternative to invasive measurements, the initial calibration may be performed by using a recording of a known continuous blood pressure output. The recording may substitute for the invasive continuous blood pressure outputs used for the initial calibration. The recording may be generated, for example, using an invasive measurement. Accordingly, PPG device 120 may be configured to provide continuous blood pressure outputs based on the initial calibration using a recording of continuous blood pressures or directly using an invasive measurement technique disclosed herein.

Such a continuous blood pressure output is an improvement over traditional devices that output blood pressure values based on greater than approximately 3 seconds per sample. For example, such traditional devices may provide as few as three blood pressure outputs over a twenty four hour period.

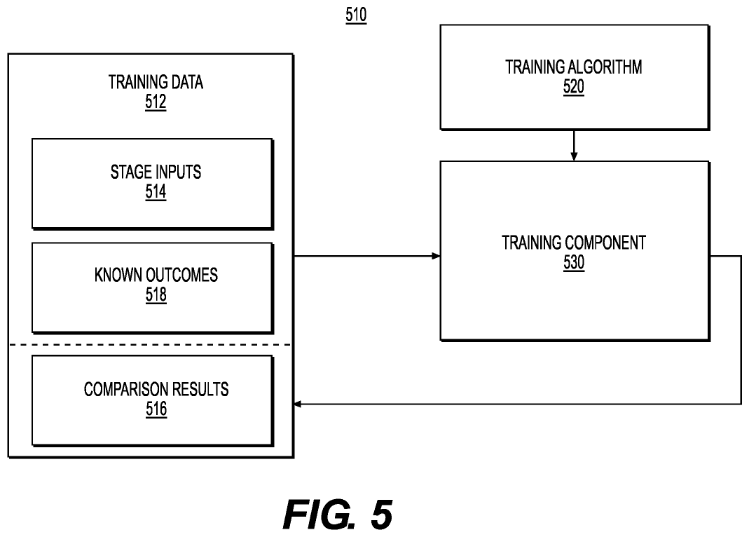

FIG. 5 depicts a flow diagram for training a machine learning model to generate a calibration factor and/or update a calibration factor, according to one or more embodiments. One or more of training data 512, stage inputs 514, known outcomes 518, comparison results 516, training algorithm 520, and training component 530 may communicate by any suitable means. One or more implementations disclosed herein may be implemented by using a trained machine learning model. A machine learning model, as disclosed herein, may be trained using environment 100 of FIG. 1A, environment 140 of FIG. 1B, environment 150 of FIG. 1C, environment 150 of FIG. 1D, environment 200 of FIG. 2A, environment 250 of FIG. 2B, environment 300 of FIG. 3A, environment 340 of FIG. 3B, environment 360 of FIG. 3C, and/or environment 400 of FIG. 4. As shown in flow diagram 510 of FIG. 5, training data 512 may include one or more of stage inputs 514 and known outcomes 518 related to a machine learning model to be trained.

Training data 512 may include historical user blood pressure, historical user medical diagnoses, or historical PPG device 120 heights. Historical blood pressure may include readings from a gold standard device, a PPG device, a data storage system, or any other suitable source. Historical user medical diagnoses may include medical records, medical conditions, and/or any other relevant medical information. Historical PPG device heights may include PPG device heights from a gold standard device, PPG device, data storage system, and/or any other suitable device or system. Training data 512 may include data from a user and/or from a plurality of users. The data from the user or plurality of users may include user height, user medical condition, or user PPG device height relative to a user heart. The stage inputs 514 may be from any applicable source including a component or set shown in FIGS. 1A, 1B, 1C, 1D, 2, 3A, 3B, 3C, and/or 4. Known outcomes 518 may be included for machine learning models generated based on supervised or semi-supervised training. An unsupervised machine learning model might not be trained using known outcomes 518. Known outcomes 518 may include known or desired outputs for future inputs similar to or in the same category as stage inputs 514 that do not have corresponding known outputs.

Training data 512 and a training algorithm 520 may be provided to a training component 530 that may apply training data 512 to training algorithm 520 to generate a trained machine learning model. According to an implementation, training component 530 may be provided comparison results 516 that compare a previous output of the corresponding machine learning model to apply the previous result to re-train the machine learning model. Comparison results 516 may be used by training component 530 to update the corresponding machine learning model. Training algorithm 520 may utilize machine learning networks and/or models including, but not limited to a deep learning network such as Deep Neural Networks (“DNN”), Convolutional Neural Networks (“CNN”), Fully Convolutional Networks (“FCN”) and Recurrent Neural Networks (“RCN”), probabilistic models such as Bayesian Networks and Graphical Models, and/or discriminative models such as Decision Forests and maximum margin methods, or the like. The output of the flow diagram 510 may be a trained machine learning model.

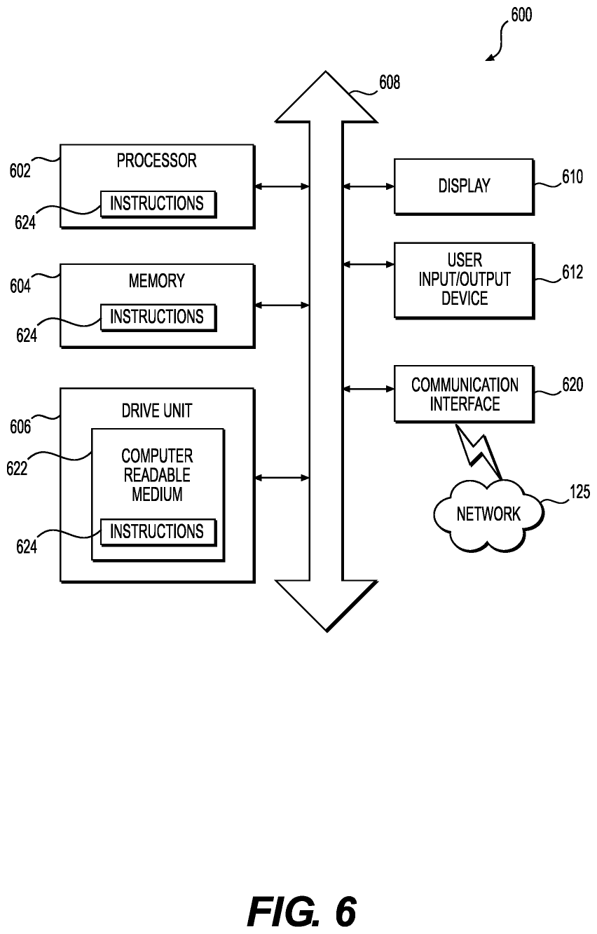

FIG. 6 is a simplified functional block diagram of a computer 600 that may be configured as a device for executing the methods of FIGS. 2 and/or 4, according to exemplary embodiments of the present disclosure. In general, any process or operation discussed in this disclosure that is understood to be computer-implementable, such as the environments and/or processes illustrated in FIGS. 1A, 1B, 1C, 1D, 2, 3A, 3B, 3C, and/or 4, may be implemented or performed by one or more processors of a computer system, such any of the systems or devices in the environment 100 of FIG. 1A, as described above. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer system. A processor may be a central processing unit (“CPU”), a graphics processing unit (“GPU”), or any suitable types of processing unit.

A computer system, such as a system or device implementing a process or operation in the examples above, may include one or more computing devices, such as one or more of the systems or devices in FIGS. 1A, 1B, 1C, 1D, 3A, 3B, and/or 3C. One or more processors of a computer system may be included in a single computing device or distributed among a plurality of computing devices. A memory of the computer system may include the respective memory of each computing device of the plurality of computing devices.

One or more of a processor 602, a memory 604, a drive unit 606, an internal communication bus 608, a display 610, a under input/output ports 612, a communication interface 620, a computer readable medium 622, instructions 624, and a network 125 may communicate by any suitable means. For example, computer 600 may be configured as PPG device 120 and/or another system according to exemplary embodiments of this disclosure. In various embodiments, any of the systems herein may be a computer 600 including, for example, data communication interface 620 for packet data communication. Computer 600 also may include a central processing unit (“CPU”) 602, in the form of one or more processors, for executing program instructions. Computer 600 may include internal communication bus 608, and storage unit 606 (such as Read-Only Memory (“ROM”), Hard Disk Drive (“HDD”), Solid-State Drive (“SSD”), etc.) that may store data on computer readable medium 622, although computer 600 may receive programming and data via network communications. Computer 600 may also have memory 604 (such as Random-Access Memory (“RAM”)) storing instructions 624 for executing techniques presented herein, although instructions 624 may be stored temporarily or permanently within other modules of computer 600 (e.g., processor 602 and/or computer readable medium 622). Computer 600 also may include input and output ports 612 and/or display 610 to connect with input and output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

While the disclosed methods, devices, and systems are described with exemplary reference to transmitting data, it should be appreciated that the disclosed embodiments may be applicable to any environment, such as a desktop or laptop computer, an automobile entertainment system, a home entertainment system, etc. Also, the disclosed embodiments may be applicable to any type of Internet protocol.

It should be understood that embodiments in this disclosure are exemplary only, and that other embodiments may include various combinations of features from other embodiments, as well as additional or fewer features. It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

1. A method for determining a blood pressure calibration factor, the method comprising:
sensing a first blood pressure by a first device when the first device is at a first height, wherein the first height is at or above a heart level of a user;
sensing a second blood pressure by the first device when the first device is at a second height, wherein the second height is at or below the heart level of the user;
generating the blood pressure calibration factor based on determining a substantially linear relationship between the first blood pressure, the first height, the second blood pressure, and the second height;
sensing a third blood pressure using a second device, when the second device is at a second device height; and
modifying the third blood pressure based on the blood pressure calibration factor and the second device height.

2. (canceled)

3. The method of claim 1, wherein the second device is a photoplethysmography (PPG) device.

4. The method of claim 1, wherein the second device is configured to detect a user blood pressure and the second device height continuously.

5. The method of claim 1, wherein the second device is configured to detect a user blood pressure using light.

6. The method of claim 1, further comprising applying at least one of a noise reduction filter or a signal amplifier after modifying the third blood pressure.

7. The method of claim 1, further comprising a machine learning model, wherein the blood pressure calibration factor is applied using the machine learning model trained to correlate the second device height with the third blood pressure to generate a machine learning output for modifying the third blood pressure.

8. The method of claim 7, wherein the machine learning model is trained to generate the machine learning output further based on additional user information selected from one or more a use medical medication information, a user medical history, a user demographic, a climate, or user hemoglobin subtype information.

9. The method of claim 8, wherein the machine learning model is trained using one or more of historical blood pressures, historical medical diagnoses, or historical device heights for a plurality of users and wherein training the machine learning model further comprises:
receiving training data including one or more of the historical blood pressure, historical medical diagnoses, or historical device heights;
receiving outcome data corrected based on one or more of the historical blood pressures, historical medical diagnoses, or historical device heights;
modifying at least one of weights, biases, or layers of a training model based on the training data and the outcome data; and
outputting the machine learning model based on the modifying at least one of weights, biases, or layers of the training model.

10. The method of claim 9, wherein the machine learning model output is individualized to each user of a plurality of users based on one or more of a user height, a medical condition, or a device height relative to a reference point.

11. The method of claim 1, wherein the blood pressure calibration factor comprises one of a linear relationship or a non-linear relationship.

12. The method of claim 1, wherein the first device is calibrated based on a column of mercury.

13. The method of claim 1, wherein the first device includes a column of mercury that detects blood pressures.

14. A system for calibrating blood pressure sensed by a photoplethysmography (PPG) device, the system comprising:
at least one memory storing instructions; and
at least one processor executing the instructions to perform a process, the at least one processor configured to:
receive a blood pressure sensed using the PPG device;
receive a PPG device height when the blood pressure is sensed; and
modify the blood pressure based on the PPG device height and a blood pressure calibration factor, wherein the blood pressure calibration factor is based on determining a substantially linear relationship between a first blood pressure sensed at a first device height and a second blood pressure sensed at a second device height, wherein the first height is at or above a heart level of a user and the second height is at or below the heart level of the user.

15. The system of claim 14, further comprising storing the blood pressure calibration factor at the at least one memory.

16. The system of claim 14, wherein the first blood pressure and the second blood pressure are sensed using one of a mercury calibrated device or a device that uses a mercury column to detect blood pressures.

17. The system of claim 14, wherein the PPG device height is one of an average height of the PPG device over a duration of sensing the blood pressure or a position of the PPG device at a predetermined time within the duration of sensing the blood pressure.

18. The system of claim 14, wherein the PPG device comprises a sensor to detect the PPG device height relative to a reference point.

19. A method for calibrating blood pressure, the method comprising:
sensing a first blood pressure using a PPG device, when the PPG device is at a first position, wherein the first position is at or above a heart level of a user;
sensing a second blood pressure using the PPG device, when the PPG device is at a second position, wherein the second position is at or below the heart level of the user;
determining a blood pressure calibration factor based on determining a substantially linear relationship between the first blood pressure, the first position, the second blood pressure, and the second position;
sensing a third blood pressure using the PPG device when the PPG device is at a PPG device position; and
modifying the third blood pressure based on the PPG device position and the blood pressure calibration factor.

20. The method of claim 19, further comprising a machine learning model, wherein the blood pressure calibration factor is applied using the machine learning model trained to correlate the PPG device position with the third blood pressure to generate a machine learning output for modifying the third blood pressure.
